(19) United States
(12) Patent Application Publication
(10) Pub. No.: US 2023/0063557 A1
(43) Pub. Date: Mar. 2, 2023

(54) MULTI-LUMEN CRYOGENIC PROBE

(71) Applicant: AtriCure, Inc., Mason, OH (US)

(72) Inventors: Saloni Jaikamal, Cincinnati, OH (US); Geni M. Giannotti, Cincinnati, OH (US); Christopher Widenhouse, Mason, OH (US); Jonathon McHale, Mason, OH (US); Stefan Stefanov, Ludlow, KY (US)

(73) Assignee: AtriCure, Inc., Mason, OH (US)

(21) Appl. No.: 17/465,079

(22) Filed: Sep. 2, 2021

(51) Int. Cl. A61B 18/02

(52) U.S. Cl. CPC A61B 18/0218; A61B 2018/00577

(57) ABSTRACT

Cryogenic probes and related methods are disclosed. An example cryogenic probe may include a distal ablation tip and an elongated, generally tubular shaft extending proximally from the ablation tip. The shaft may include a flexible thermoplastic body, a supply conduit extending through the body, and/or an exhaust conduit extending through the body. At least one of the supply conduit and the exhaust conduit may be embedded within the body.

### INTRODUCTION TO THE INVENTION

The present disclosure is directed to cryogenic devices, and, more specifically, to cryogenic probes having a flexible shaft connected to an ablation tip, and related methods.

The present disclosure contemplates that cryogenic devices, such as cryogenic probes, may be used in various medical and surgical procedures. Generally, cryogenic probes may be used to apply extremely cold temperatures to a target tissue. Cryogenic probes may be used for cryoablation and/or cryoanalgesia, for example.

The present disclosure contemplates that some cryogenic probes may be supplied with one or more cryogenic fluids, which may be used to cool a tissue-contacting portion, such as an ablation tip. Some cryogenic probes may include supply conduits, which convey cryogenic fluid to the ablation tip, and exhaust conduits, which convey used cryogenic fluid away from the ablation tip. Some cryogenic probes may utilize cryogenic fluids supplied at high pressures. For example, some cryogenic probes employing Joule-Thompson expansion in the ablation tip may receive liquid nitrous oxide at up to about 1200 psi and about room temperature and/or may exhaust the nitrous oxide as a gas or mixed phase of gas and liquid at about 45 psi and about −90° F. The cryogenic probe and associated conduits and connectors may be designed to withstand such pressures and temperatures.

The following patent references may provide context for the present disclosure and are incorporated by reference herein in their entireties: U.S. Pat. No. 8,915,908, U.S. Patent Application Publication No. 2020/0022745, and U.S. Patent Application Publication No. 2020/0085485.

The present disclosure contemplates that while cryogenic probes may utilize extremely cold temperatures to achieve desired effects at desired locations, exposure of other locations to extremely cold temperatures may cause undesired effects. For example, it may be desirable for the ablation tip of a cryogenic probes to be extremely cold while the shaft on which the tip is disposed remains above a tissue-ablating temperature. The present disclosure provides methods and apparatus that improve the ability of cryogenic probes to cool an ablation tip to a desired temperature while maintaining other portions of the cryogenic probes at warmer temperatures to reduce the likelihood of freezing non-target tissues.

While known cryogenic devices have been used safely and effectively to perform cryosurgical procedures, improvements in the construction and operation of cryogenic probes may be beneficial for users (e.g., surgeons) and patients. The present disclosure includes various improvements which may enhance the construction, operation, and methods of use of cryogenic probes.

It is an aspect of the present disclosure to provide a cryogenic probe including a distal ablation tip and/or an elongated, generally tubular shaft extending proximally from the ablation tip. The shaft may include a flexible thermoplastic body, a supply conduit extending through the body, the supply conduit being configured to convey cryogenic fluid distally to the ablation tip, and/or an exhaust conduit extending through the body, the exhaust conduit being configured to convey spent cryogenic fluid proximally from the ablation tip. The supply conduit and/or the exhaust conduit may be embedded within the body.

In a detailed embodiment, the exhaust conduit may be disposed generally centrally within the body. The supply conduit may be generally helically shaped. The generally helically shaped supply conduit may be disposed radially around the generally centrally disposed exhaust conduit. The exhaust conduit may be generally concentrically disposed within the supply conduit. The supply conduit may be generally concentrically disposed within the body.

In a detailed embodiment, the supply conduit may be embedded in the body and/or the exhaust conduit may be embedded in the body. The supply conduit and the exhaust conduit may be disposed in the body in a generally parallel and spaced apart arrangement.

In a detailed embodiment, the shaft may include at least one auxiliary lumen extending generally longitudinally through the body.

In a detailed embodiment, at least one of the supply conduit and the exhaust conduit may include a reinforcing structure. The reinforcing structure may include a generally tubular braid reinforcement. The braid reinforcement may be constructed from at least one of stainless steel, nylon, high density polyethylene, polyethylene terephthalate, carbon fibers, and/or poly para-aramid.

It is an aspect of the present disclosure to provide a cryogenic surgical system including a cryogenic probe as described above and/or a cryogenic module configured to be operatively coupled to the cryogenic probe to supply cryogenic fluid to the cryogenic probe.

It is an aspect of the present disclosure to provide a method of making a cryogenic probe including providing an elongated, generally tubular shaft. The shaft may include a flexible thermoplastic body, a supply conduit extending through the body, the supply conduit being configured to convey cryogenic fluid, and/or an exhaust conduit extending through the body, the exhaust conduit being configured to convey spent cryogenic fluid. The supply conduit and/or the exhaust conduit may be embedded within the body. The method may include attaching an ablation tip distally on the shaft so that an internal cavity of the ablation tip fluidically interposes the supply conduit and the exhaust conduit.

In a detailed embodiment, the method may include attaching a handle proximally on the shaft. Providing the elongated, generally tubular shaft may include extruding the body. Extruding the body may include embedding the supply conduit and/or the exhaust conduit therein. Extruding the body may include forming at least one auxiliary lumen extending generally longitudinally through the body.

In a detailed embodiment, the exhaust conduit may be disposed generally centrally within the body. The supply conduit may be generally helically shaped. The generally helically shaped supply conduit may be disposed radially around the generally centrally disposed exhaust conduit. The exhaust conduit may be generally concentrically disposed within the supply conduit.

In a detailed embodiment, the supply conduit and the exhaust conduit may be disposed in the body in a generally parallel and spaced apart arrangement.

In a detailed embodiment, the supply conduit and/or the exhaust conduit may include a reinforcing structure. The reinforcing structure may include a generally tubular braid reinforcement.

### BRIEF DESCRIPTION OF THE DRAWINGS

Example embodiments are described in conjunction with the accompanying drawing figures in which:

FIG. 1 is a perspective view of an example cryogenic probe depicted in various bending positions;

FIG. 2 is a partial longitudinal cross-section view of a distal portion of the cryogenic probe of FIG. 1;

FIG. 3 is a lateral cross-section view of an alternative example shaft;

FIG. 4 is a longitudinal cut-away view of the alternative example shaft; and

FIG. 5 is a longitudinal cut-away view of an alternative example shaft; all in accordance with at least some aspects of the present disclosure.

### DETAILED DESCRIPTION

Example embodiments according to the present disclosure are described and illustrated below to encompass devices, methods, and techniques relating to cryogenic devices, such as a cryogenic probe having a flexible shaft connected to an ablation tip, and related methods. Of course, it will be apparent to those of ordinary skill in the art that the embodiments discussed below are examples and may be reconfigured without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure. Unless explicitly stated otherwise, any feature or function described in connection with any example embodiment may apply to other example embodiments, and repeated description of similar features and functions is omitted for brevity.

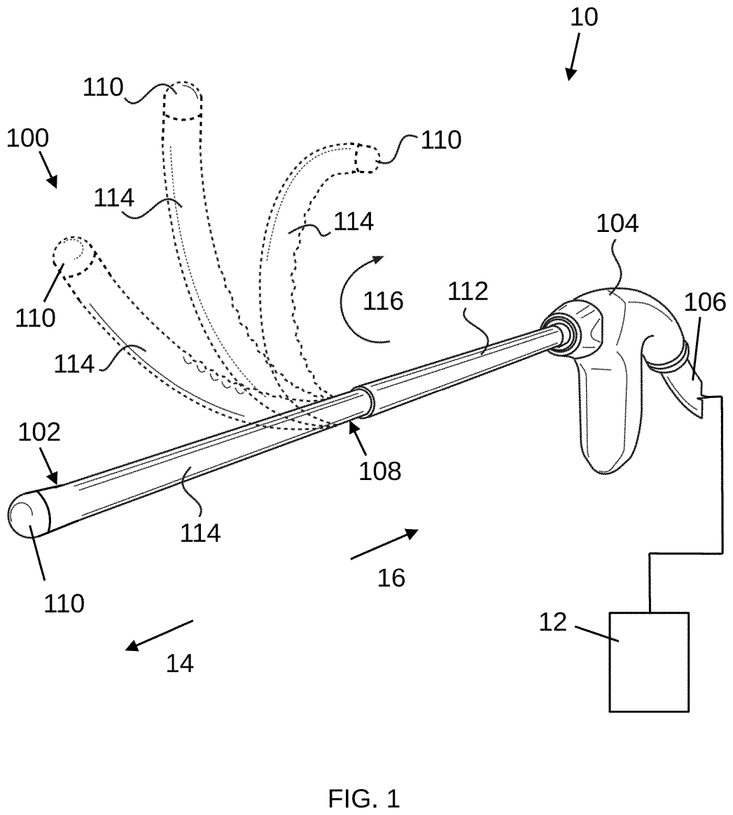

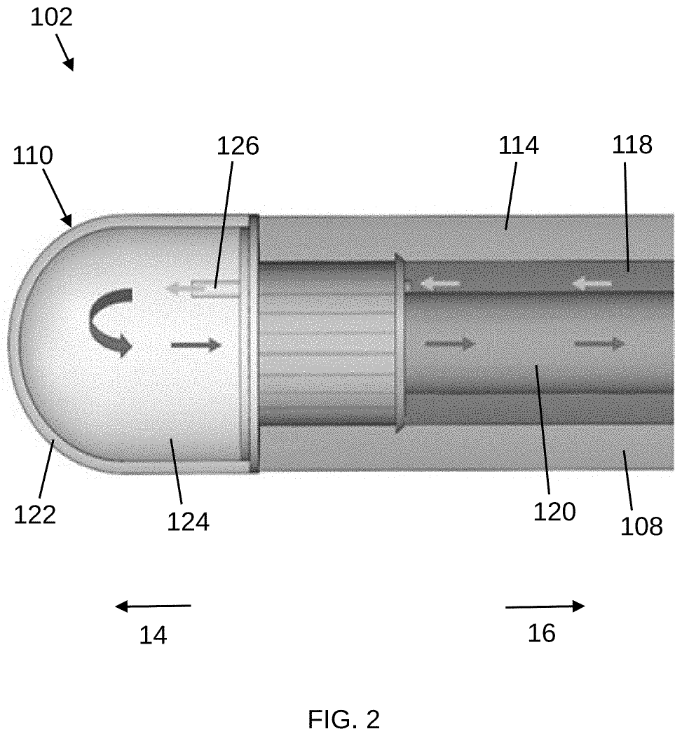

FIG. 1 is a perspective view of an example cryogenic probe 100 depicted in various bending positions and FIG. 2 is a partial longitudinal cross-section view of a distal portion 102 of the cryogenic probe 100, all according to at least some aspects of the present disclosure. The cryogenic probe 100 may be a part of a cryogenic surgical system 10, which may include a cryogenic module 12. The cryogenic probe 100 may be operatively coupled to the cryogenic module 12, which may be configured to supply cryogenic fluid to and/or receive cryogenic fluid from the cryogenic probe 100, as well as provide control functions. Example cryogenic fluids may include one or more of nitrous oxide, argon, carbon dioxide, and/or phase change fluids (e.g., liquid nitrogen).

For clarity, the following description references a distal direction 14 and a proximal direction 16. The proximal direction 16 may be generally opposite the distal direction 14. As used herein, “distal” may refer to a direction generally away from an operator of a system or device (e.g., a surgeon), such as toward the distant-most end of a device that is inserted into a patient's body. As used herein, “proximal” may refer to a direction generally toward an operator of a system or device (e.g., a surgeon), such as away from the distant-most end of a device that is inserted into a patient's body. It is to be understood, however, that example directions referenced herein are merely for purposes of explanation and clarity, and should not be considered limiting.

Referring to FIGS. 1 and 2, the example cryogenic probe 100 may include a handle 104, which may be configured to be grasped by a user (e.g., surgeon) and/or a robotic device (e.g., a surgical robot). More generally, the handle 104 may comprise any structure that may be configured to be secured, held, and/or manipulated to position or restrain the cryogenic probe 100, regardless of whether it may be utilized by a human (e.g., surgeon or assistant), robot, mechanical device, etc. The handle 104 may be configured to connect to the cryogenic module 12 using one or more cryogenic fluid lines 106 (e.g., cryogenic fluid supply and/or exhaust lines). The cryogenic probe 100 may include an elongated, generally tubular shaft 108 disposed generally distally on the handle 104. The distal portion 102 of the cryogenic probe 100 may include an ablation tip 110, which may be disposed distally on the shaft 108.

In some example embodiments, the shaft 108 may include a first, proximal portion 112 and/or a second, distal portion 114. In some example embodiments, the proximal portion 112 of the shaft 108 may be generally rigid and/or generally elastically deformable. For example, the proximal portion 112 of the embodiment illustrated in FIG. 1 may be configured to retain and/or return to a generally straight shape. In some example embodiments, the distal portion 114 of the shaft 108 may be generally flexible. As used herein, “flexible” may describe a component that is elastically and/or plastically deformable when subject to forces consistent with normal, intended use of the device. For example, a flexible component may be bendable and/or malleable. In some example embodiments, the distal portion 114 of the shaft 108 may be configured for bending at an angle 116 relative to the handle 104 and/or the proximal portion 112 of the shaft 108. Although the example embodiment illustrated in FIGS. 1 and 2 may include a shaft 108 including both a generally rigid portion 112 and a generally flexible portion 114, various alternative example embodiments may include shafts including only one or more generally flexible portions, only one or more generally rigid portions, or any combination of generally flexible portions and/or generally rigid portions, in any arrangement.

Referring to FIG. 2, in some example embodiments, the shaft 108 may include one or more internal conduits configured to direct cryogenic fluid to and/or from the handle 104 (FIG. 1). For example, the distal portion 114 of the shaft 108 may include a supply conduit 118 configured to convey cryogenic fluid to the ablation tip 110 and/or an exhaust conduit 120 configured to convey spent cryogenic fluid from the ablation tip 110. As used herein, “spent cryogenic fluid” may refer to cryogenic fluid that has exited the ablation tip 110 (e.g., into the exhaust conduit 120), regardless of its phase, temperature, or pressure and regardless of whether it may be capable of further cooling.

In some example embodiments, the ablation tip 110 may include a wall 122 at least partially defining an internal cavity 124. The wall 122 may at least partially define a generally rounded shape, or any other shape as desired for engagement with a target anatomy. The ablation tip 110 may include a nozzle 126 (e.g., an orifice) through which cryogenic fluid from the supply conduit 118 enters the internal cavity 124. The internal cavity 124 may be fluidically coupled to the exhaust conduit 120. Thus, the internal cavity 124 of the ablation tip 110 may fluidically interpose the supply conduit 118 and the exhaust conduit 124.

In operation, cryogenic fluid supplied from the cryogenic module 12 (FIG. 1) may flow through the supply conduit 118 and the nozzle 126 into the internal cavity 124 of the ablation tip 110. Generally, the cryogenic fluid exiting the nozzle 126 may be at a higher pressure upstream from the nozzle 126 and may be allowed to expand downstream of the nozzle 126 within the cavity 124 at a significantly lower pressure, thereby creating a Joule-Thompson expansion and significantly lowering the temperature of the cryogenic fluid and the ablation tip 110. In some example embodiments utilizing nitrous oxide as a cryogenic fluid, the nitrous oxide may be supplied as a liquid at a temperature of about 27 C and a pressure of about 800 psi upstream of the nozzle 126, and may comprise a gaseous phase or a mixed phase of gas and liquid at approximately 45 psi and −68 C within the ablation tip 110 internal cavity 124. As Joule-Thompson expansion continues occurring within the ablation tip 110, the wall 122 becomes cooled enough for use in an ablation procedure, such as by bringing the ablation tip 110 into contact with tissue to be ablated. By way of example, depending upon the cryogen fluid utilized, exemplary flow rates for cryogenic fluid through the nozzle 126 may range between approximately fifteen to greater than one hundred cubic centimeters per minute.

In the example embodiment illustrated in FIG. 2, the exhaust conduit 120 may be generally concentrically disposed within the supply conduit 118 and/or the supply conduit 118 may be disposed generally concentrically within the distal portion 114 of the shaft 108. As used herein, “concentric” may describe components which are arranged so that they have a common center point. In alternative example embodiments, the conduits 118, 120 may be disposed within one another and/or within the shaft 108 non-concentrically. In some example embodiments, the cryogenic fluid flowing to the ablation tip 110 may be substantially warmer than the spent cryogenic fluid flowing from the ablation tip 110. Accordingly, disposing the exhaust conduit 120 generally within the supply conduit 118 (concentrically or non-concentrically) may allow the supply conduit 118 and/or the relatively warmer cryogenic fluid therein to insulate tissues near the shaft 108 from the cold spent cryogenic fluid in the exhaust conduit 120.

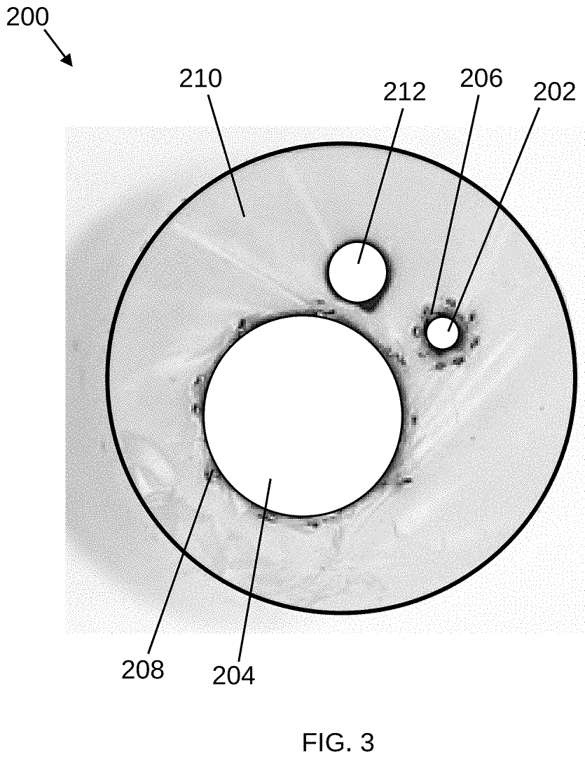

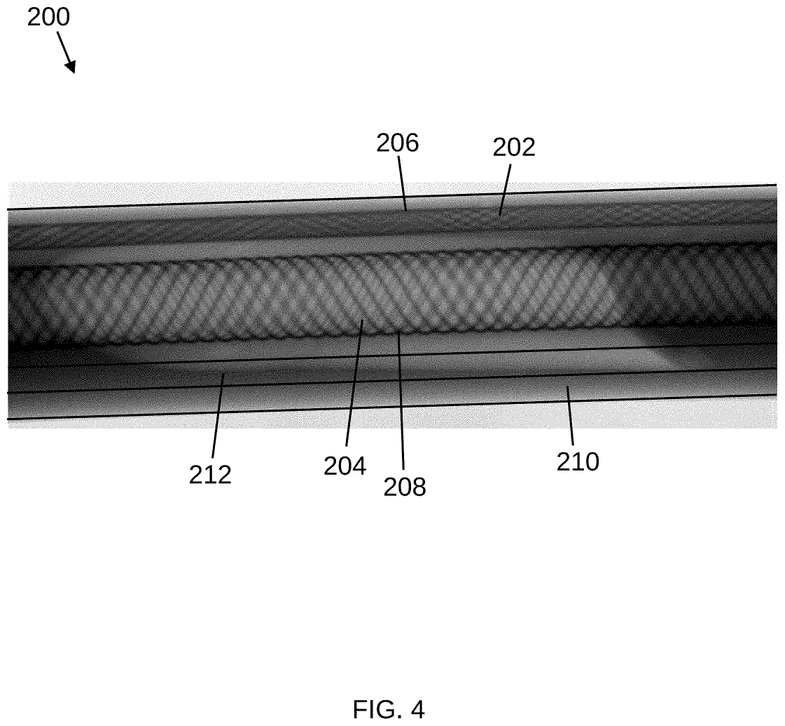

FIG. 3 is a lateral cross-section view of an alternative example shaft 200 and FIG. 4 is a longitudinal cut-away view of the alternative example shaft 100, all according to at least some aspects of the present disclosure. In some example embodiments, the alternative example shaft 200 may be utilized in place of the shaft 108 and/or the distal portion 114 of the shaft 108 in the example cryogenic probe 100 described above with reference to FIGS. 1 and 2.

Referring to FIGS. 3 and 4, in some example embodiments, the shaft 200 may include a supply conduit 202 configured to convey cryogenic fluid to an ablation tip and/or an exhaust conduit 204 configured to convey spent cryogenic fluid from the ablation tip. In the example embodiment illustrated in FIGS. 3 and 4, the supply conduit 202 and the exhaust conduit 204 are arranged non-concentrically, generally straight (e.g., longitudinally), generally in parallel, and/or generally spaced apart. In alternative example embodiments, the supply conduit 202 and the exhaust conduit 204 may be arranged one within the other (e.g., concentrically or non-concentrically) and/or may be arranged substantially adjacent (e.g., in contact or nearly in contact) with one another.

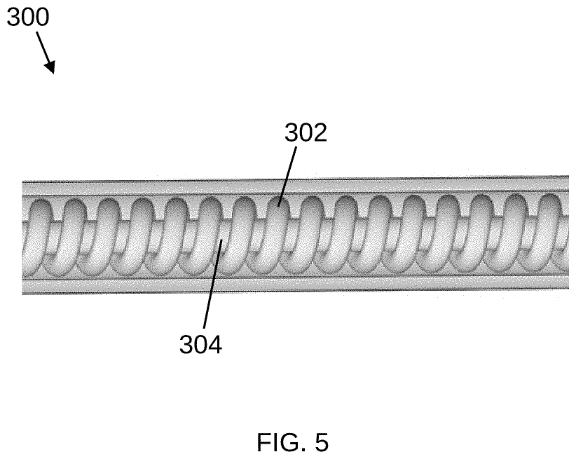

In some alternative example embodiments, one or more of the conduits 202, 204 may be arranged in a generally spiral (e.g., helical) configuration. FIG. 5 is a longitudinal cut-away view of an alternative example shaft 300, according to at least some aspects of the present disclosure. Shaft 300 may include a supply conduit 302 generally in the form of a helix disposed radially around a generally longitudinally straight exhaust conduit 304. As shown in FIG. 5, in some example embodiments, the exhaust conduit 304 may be disposed generally centrally within the shaft 300.

Referring to FIGS. 3 and 4, in some example embodiments, one or more of the supply conduit 202 and/or the exhaust conduit 204 may include a reinforcing structure, such as a generally tubular braid, mesh, and/or other type of reinforcing structure. For example, the reinforcing structures may include braid reinforcements 206, 208, and may be constructed from a variety of materials such as stainless steel, nylon, high density polyethylene, polyethylene terephthalate, carbon fibers, poly para-aramid (Kevlar, for example), and/or the like. Unlike extruded catheters utilized for relatively low-pressure applications, the reinforcing structures may allow various example embodiments to be used with cryogenic fluids at relatively high pressures (e.g., 600-1000 psi).

In some example embodiments, the shaft 200 may include a body 210 at least partially containing the conduits 202, 204. For example, the shaft 200 may include a body 210 having a generally circular cross section disposed around the conduits 202, 204. In some example embodiments, the body 210 may be formed from extruded thermoplastic. In the example embodiment illustrated in FIGS. 3 and 4, the conduits 202, 204 are disposed non-concentrically within the body 210. In some alternative example embodiments, one or more of the conduits 202, 204 (or other lumens) may be positioned concentrically within the body 210. For example, the exhaust conduit 204 may be positioned generally centrally within the body. Such an arrangement may be utilized when it is desirable to increase the insulation between a cold exhaust conduit 204 and tissues adjacent to the shaft 200 by increasing the thickness of the portion of the body 210 therebetween.

The body 210 may be partially or completely substantially solid and/or may include one or more voids and/or lumens in addition to the one or more conduits 202, 204. For example, the body 210 may include one or more auxiliary lumens 212. For example, one or more auxiliary lumens 212 may extend longitudinally the entire length of the shaft 200 and/or one or more auxiliary lumens 212 may extend only partway through the length of the shaft 200.

In some example embodiments, an auxiliary lumen 212 may be used to route a component or instrument generally between a proximal portion of the shaft 200 and a distal portion of the shaft 200. For example, thermocouple wires may be routed through the auxiliary lumen 212. In some example embodiments, one or more auxiliary lumens 212 may be used to convey a fluid (e.g., a fluid other than the cryogenic fluid) longitudinally along the shaft 200. For example, a warming fluid (e.g., air, water, saline, etc.) may be conveyed from a proximal portion of the shaft 200 to a distal portion of the shaft 200. Some example embodiments may include one or more additional auxiliary lumens, which may act as a return pathway for such warming fluids. In some example embodiments, one or more auxiliary lumens 212 or voids may act as insulation, such as between one or more of the conduits 202, 204 and tissues adjacent to the shaft 200.

In the example embodiment illustrated in FIGS. 3 and 4, the auxiliary lumen 212 may not include a reinforcing structure. In alternative example embodiments, one or more auxiliary lumens 212 may include reinforcing structures, such as braid reinforcements similar to the braid reinforcements 206, 208 associated with the conduits 202, 204.

In some example embodiments, materials for various components of the shaft may be selected or configured to provide desired insulation and/or flexibility characteristics. For example, materials, such as thermoplastics, may be used to form various components of the shaft 200. Some example embodiments may include laser cuts (e.g., to increase flexibility) and/or wire braid reinforcements (e.g., to increase strength).

Some example embodiments according to at least some aspects of the present disclosure may be fabricated by one or more extrusion processes. For example, the thermoplastic body 210 may be extruded around previously formed braid reinforcements 206, 208. In some example embodiments, additional lumens or voids (e.g., auxiliary lumen 212) may be formed as the body 210 is extruded. In some example embodiments, the resulting shaft may comprise a single-piece structure including a one or more embedded conduits and/or lumens, one or more of which may be reinforced. In other words, some example embodiments may comprise a multi-lumen shaft including a single tube extruded with multiple conduits/lumens embedded therein.

Some example embodiments according to at least some aspects of the present disclosure may include shaft elements that provide improved usability relative to other cryogenic probes, such as cryogenic probes utilizing relatively bulky external insulative materials. For example, some example embodiments may be generally more flexible and/or smaller in diameter than other cryogenic probes, which may improve use in connection with robotic instrumentation and/or which may facilitate improved access to difficult anatomy, such as during open surgical procedures.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope as defined by the following claims. Likewise, it is to be understood that it is not necessary to meet any or all of the identified advantages or objects disclosed herein in order to fall within the scope of the claims, since inherent and/or unforeseen advantages may exist even though they may not have been explicitly discussed herein.

What is claimed is:

1. A cryogenic probe comprising:
a distal ablation tip; and
an elongated, generally tubular shaft extending proximally from the ablation tip;
wherein the shaft comprises
a flexible thermoplastic body,
a supply conduit extending through the body, the supply conduit being configured to convey cryogenic fluid distally to the ablation tip, and
an exhaust conduit extending through the body, the exhaust conduit being configured to convey spent cryogenic fluid proximally from the ablation tip; and
wherein at least one of the supply conduit and the exhaust conduit is embedded within the body.

2. The cryogenic probe of claim 1, wherein the exhaust conduit is disposed generally centrally within the body.

3. The cryogenic probe of claim 2,
wherein the supply conduit is generally helically shaped; and
wherein the generally helically shaped supply conduit is disposed radially around the generally centrally disposed exhaust conduit.

4. The cryogenic probe of claim 2, wherein the exhaust conduit is generally concentrically disposed within the supply conduit.

5. The cryogenic probe of claim 4, wherein the supply conduit is generally concentrically disposed within the body.

6. The cryogenic probe of claim 1,
wherein the supply conduit is embedded in the body;
wherein the exhaust conduit is embedded in the body.

7. The cryogenic probe of claim 6, wherein the supply conduit and the exhaust conduit are disposed in the body in a generally parallel and spaced apart arrangement.

8. The cryogenic probe of claim 1, wherein the shaft further comprises at least one auxiliary lumen extending generally longitudinally through the body.

9. The cryogenic probe of claim 1, wherein at least one of the supply conduit and the exhaust conduit comprises a reinforcing structure.

10. The cryogenic probe of claim 9, wherein the reinforcing structure comprises a generally tubular braid reinforcement.

11. The cryogenic probe of claim 10, wherein the braid reinforcement is constructed from at least one of stainless steel, nylon, high density polyethylene, polyethylene terephthalate, carbon fibers, and poly para-aramid.

12. A cryogenic surgical system comprising:
the cryogenic probe of claim 1; and
a cryogenic module configured to be operatively coupled to the cryogenic probe to supply cryogenic fluid to the cryogenic probe.

13. A method of making a cryogenic probe, the method comprising:
providing an elongated, generally tubular shaft comprising
a flexible thermoplastic body,
a supply conduit extending through the body, the supply conduit being configured to convey cryogenic fluid, and
an exhaust conduit extending through the body, the exhaust conduit being configured to convey spent cryogenic fluid,
wherein at least one of the supply conduit and the exhaust conduit is embedded within the body; and
attaching an ablation tip distally on the shaft so that an internal cavity of the ablation tip fluidically interposes the supply conduit and the exhaust conduit.

14. The method of claim 13, further comprising attaching a handle proximally on the shaft.

15. The method of claim 13,
wherein providing the elongated, generally tubular shaft comprises extruding the body; and
wherein extruding the body comprises embedding the at least one of the supply conduit and the exhaust conduit therein.

16. The method of claim 15, wherein extruding the body comprises forming at least one auxiliary lumen extending generally longitudinally through the body.

17. The method of claim 13, wherein the exhaust conduit is disposed generally centrally within the body.

18. The method of claim 17,
wherein the supply conduit is generally helically shaped; and
wherein the generally helically shaped supply conduit is disposed radially around the generally centrally disposed exhaust conduit.

19. The method of claim 17, wherein the exhaust conduit is generally concentrically disposed within the supply conduit.

20. The method of claim 13, wherein the supply conduit and the exhaust conduit are disposed in the body in a generally parallel and spaced apart arrangement.

21. The method of claim 13, wherein at least one of the supply conduit and the exhaust conduit comprises a reinforcing structure.

22. The method of claim 21, wherein the reinforcing structure comprises a generally tubular braid reinforcement.
